(19) United States
(12) Patent Application Publication
(10) Pub. No.: US 2005/0187428 A1
(43) Pub. Date: Aug. 25, 2005

(54) Intra-urethral sphincter and method and means for anchoring it in a urethra

(76) Inventor: Ase Rinman, Molndal (SE)

Correspondence Address: Gauthier & Connors LLP, Suite 3300, 225 Franklin Street, Boston, MA, 02110, US

(21) Appl. No.: 11/046,488

(22) Filed: Jan. 28, 2005

Related U.S. Application Data
(63) Continuation of application No. PCT/SE03/01230, filed on Jul. 21, 2003.

(30) Foreign Application Priority Data
Jul. 29, 2002 (SE) 0202334-9

(51) Int. Cl. A61F 2/00

(52) U.S. Cl. 600/29

(57) ABSTRACT

An artificial sphincter comprises an anchoring element in form of a shape memory coil, which has a transition temperature, which is significantly higher than the human body temperature of about 37° C. At lease a portion of the cylindrical sphincter body is enclosed by the coil. The coil to be threaded onto threads provided at the exterior mantle face of the body. Also disclosed is a method for inserting and anchoring the sphincter in an urethra and a device for inserting and anchoring the sphincter which comprises a proximal end portion of about cylindrical shape, means for coupling with the sphincter disposed at the proximal end of said proximal end portion and being operable from a distal end portion thereof, a channel disposed within the proximal end portion which has an opening at the proximal end thereof for adducing an aqueous fluid of a temperature above 37° C. to the shape memory metal coil.

### FIELD OF THE INVENTION

The present invention relates to an artificial sphincter, in particular an artificial intra-urethral sphincter, and to a method and means for its insertion into a soft tissue vessel, in particular a urethra, where it is non-surgically and removably anchored.

### BACKGROUND OF THE INVENTION

Urinary incontinence is a common problem in men and women. Various ways for coping with its manifestations are known in the art. Irregular urination or diurea, particularly such as associated with diabetes insipidus and nocturnal enuresis, can be treated by medication, for instance by administration of the vasopressin hormone analogue desmopressin. Other conditions, such as conditions in which the urethral sphincter or its nervous control are damaged, require physical means to retain the urine in the bladder over selected periods of time from which it is emptied when the patients so decides. Means for retaining the urine in the bladder comprise urethral valves which are controllable from outside of the urethra and which may be permanently implanted or temporarily inserted into the urethra. Permanent implantation requires surgery. The advantage with temporarily inserted urethral valves, that is, artificial intra-urethral sphincters, in the following called artificial sphincters, is that they can be removed if not properly working and exchanged for new ones which may be of same or different design; it is to this kind of artificial urethral valves to which the present invention relates.

U.S. Pat. No. 6,022,312 A discloses an endosphincter that need not be implanted, and a method for inserting it into a urethra and anchoring it there. The known sphincter comprises a retaining part and a valve body that can be secured by the retaining part in the urethra. The retaining part, which may be of a shape memory alloy in form of a Coil spring, has a cylindrical main section with a first diameter D and a likewise cylindrical end section with a smaller second diameter d at one Of its ends. The sections are permanently connected by an expanding retaining area. The transition temperature of the shape memory alloy of the retaining part is significantly lower than the human body temperature of about 37° C.

EP 0 666 065 discloses a urethral stent capable of adopting different forms is disclosed and comprising a helical body to be inserted in a vessel or a urethra or the similar.

U.S. Pat. No. 5,512,032 discloses an improved self-contained automatic bladder control device including a valve assembly mount arranged for releasable receiving a valve assembly. However, this publication provides no knowledge or hint that the cylindrical valve holder should have an expandable diameter. By having an expandable diameter the valve holder and its valve can be placed in a safe and secure way in the urethra which is essential for a complete function, as otherwise “valve incontinence” may occur, which thus, in no way, solves the problem as the valve assembly is set to solve.

### PROBLEMS OF THE INVENTION

A problem with removable urethral sphincters known in the art is to secure them temporarily in a selected portion of the urethra in a manner that does not jeopardize their removal at a later time.

Another problem with urethral sphincters of the aforementioned kind known in the art is that their design, including the design of the corresponding securing means, is not well adapted to physiological conditions in men and women.

Still another problem with such urethral sphincters is that their control by the patient is far from optimal and may even fail.

Further problems with urethral sphincters known in the art will become evident from the following short description of the invention, preferred embodiments thereof illustrated in a drawing, and the appended claims.

### SHORT DESCRIPTION OF THE INVENTION

The present invention departs from the anchoring of a urethral sphincter by a shape memory coil spring disclosed in U.S. Pat. No. 6,022,312 A.

The present invention is based on the insight that an anchoring element in form of a shape memory coil spring may advantageously have a transition temperature which is significantly higher than the human body temperature of about 37° C., such as a temperature of above 40 C, preferably of from 40° C. to 48° C., most preferred of from 41° to 45° C. This prevents the expansion of the shape memory metal coil spring to be effected by the body temperature of the patient and allows the insertion of the assembly into a selected position of the urethra in no hurry. For the proper function of sphincter of the invention it is important that the sphincter is placed in an appropriate portion of the urethra, as explained below in greater detail. When the sphincter has been properly positioned within the urethra, at least a portion of the shape memory metal coil spring is made to expand by adducing an aqueous fluid having a temperature of at least the transition temperature of the shape memory metal coil and contacting the coil with it until the desired expansion has taken place. Preferably the aqueous fluid is made to flow through the portion of the urethra where artificial sphincter has been positioned, and past the artificial sphincter into the bladder.

According to the invention is thus disclosed an artificial urethral sphincter comprising a retaining part in form of a coil of shape memory metal or other suitable shape memory material and a valve body which is at least partially enclosed by the retaining part and in a fixed relationship with it, the transition temperature of the shape memory metal being substantially above the body temperature of the subject into the urethra of which the sphincter is to be inserted. It is preferred for the transition temperature to be 40° C. and above, more preferred from 40° C. to 48° C., most preferred from 41° C. to 45° C. This temperature is high enough to prevent the shape memory metal coil to expand unintended before having been positioned in the desired position of the urethra but low enough to prevent irreversible damage of the urethra and/or the bladder.

According to first advantageous aspect of the invention, the coil of shape memory metal comprises one or several sections, which are capable of expanding at the transition temperature and above, and one or several sections, which are not capable of expanding at that temperature. Methods of how to manufacture shape memory metal coils of this kind are known in the art; see, for instance, D M Brunette et al., Titanium in Medicine, Springer Verl., Berlin 2002, p. 54- and literature cited therein. Shape memory metal coils of this kind are also found in commerce (MEMOKATH nickeltitanium stents manufactured by ENGINEERS & DOCTORS A/S, Kvistgård, Denmark). It is particularly preferred for the shape memory metal coil of this invention to have two expandable sections separated by a non-expandable section. It is also preferred to arrange two or more separate shape memory metal coils at the valve body.

According to a second advantageous aspect of the invention, the valve body or housing is of a generally cylindrical shape and of a diameter which is close to the inner diameter of at least a section of the shape memory metal coil, so as to make the valve body and the shape memory metal coil abut each other. It is particularly preferred for the valve body to have at its exterior mantle face threaded helical groves the pitch of which corresponds to the pitch of the shape memory metal coil, so as to allow the coil to be screwed or wound on it. It is also preferred for the shape memory metal coil in such abutting disposition to be slightly expanded in a radial direction, that is, to abut the valve body with a certain force. The shape memory metal coil may of course also be permanently fixed to the valve body at a non-expandable section of it but this is not preferred. Surprisingly, the coil of shape memory metal, when partially or fully expanded in situ, provides good sealing between the urethral wall and the valve body.

According to a third advantageous aspect of the invention the shape memory metal coil is of a nickel-titanium, alloy (equiatomic intermetallic compound TiNi) of an martensite/austenite transition temperature of more than 37° C., preferably 40° C. and above more preferred from 40° C. to 48°, most preferred of from 41 to 45° C.

According to the present invention is also disclosed a method for inserting an artificial sphincter comprising a coil of shape memory metal into a urethra and anchoring it there. The method utilizes an insertion device which forms part of the invention and will be described below in greater detail. The method of the invention comprises coupling an artificial sphincter including a shape memory metal coil comprising a coil section having a transition temperature above the normal human body temperature of about 37° C. to an oblong insertion device at one end thereof, inserting the artificial sphincter by means the insertion device into a urethra until a desired position is reached, adducing an aqueous fluid of a temperature equal to or higher than the transition temperature of the coil section through the insertion device to make the coil expand, thereby anchoring the artificial sphincter with the coil in the urethra, releasing the sphincter from the insertion device and withdrawing the insertion device from the urethra.

More specifically, the method of the invention comprises the following steps:
providing an artificial sphincter of about cylindrical form comprising a coil of shape memory metal having a transition temperature above the normal human body temperature of about 37° and at least partially surrounding and holding the sphincter;
coupling a distal end portion of the sphincter to a coupling means disposed at the proximal end of an oblong insertion device having a proximal end portion of about cylindrical form which comprises an axial channel for transport of fluid, the coupling means thereby holding the sphincter in a disposition in which the cylinder axes of the sphincter and the valve body coincide;
inserting the sphincter and the proximal portion of the insertion device into the urethra, thereby disposing the sphincter in a desired position in the urethra;
adducing an aqueous fluid into the urethra by said channel so as to make the fluid flow along the urethral wall and past the sphincter into the bladder, the fluid having a temperature above the critical point of the shape memory metal coil, thereby making a portion or portions of the coil expand in a radial direction;
releasing the insertion means from the sphincter; and
withdrawing the insertion means.

According to the present invention is also disclosed an oblong insertion and anchoring device which comprises a proximal end portion of about cylindrical shape, a means for sealing the external urethral opening, a means for coupling an artificial sphincter disposed at the proximal end of said proximal end portion and being operable from a distal end portion, a channel disposed within said proximal end portion and having an opening at the proximal end thereof, the channel being adapted for adducing an aqueous fluid of a temperature above the normal human body temperature of about 37° C. to the shape memory metal coil. It is preferred for the sealing means to comprise an external sealing flange set off from said proximal end by from about 10 mm to about 80 mm. The device of claim 12, wherein the coupling means comprises two or more flexible fingers disposed in said channel. The proximal portions of the fingers are preferably independently displaceable in an axial direction and in a radial direction. It is also preferred for the proximal finger portions to be capable of being displaced in an axial direction from a position within the channel to a position proximally outside of the channel. Preferably the radial displacement of the proximal finger portions is arranged for in their position proximally outside of the channel. It is also advantageous for the means for operation of the coupling means to comprise a rod disposed in the channel. Preferably the rod is disposed in the channel radially inwardly of said flexible fingers. At its proximal end the rod may comprises a slanting face arranged for co-operation with inwardly facing faces of the flexible fingers so as to Push them radially outwards by displacing the rod in a proximal direction.

### SHORT DESCRIPTION OF THE FIGURES

The invention will now be explained in more detail by reference to a preferred embodiment illustrated in a drawing in which is shown in:

FIG. 1 an artificial sphincter of the invention comprising a shape memory metal coil, in a closed position (C) and in an axial section;

FIG. 2 an insertion device of the invention, in an axial section;

FIG. 3 the artificial sphincter of FIG. 1 coupled with the insertion device of FIG. 2, in an open position (0) and in the same view;

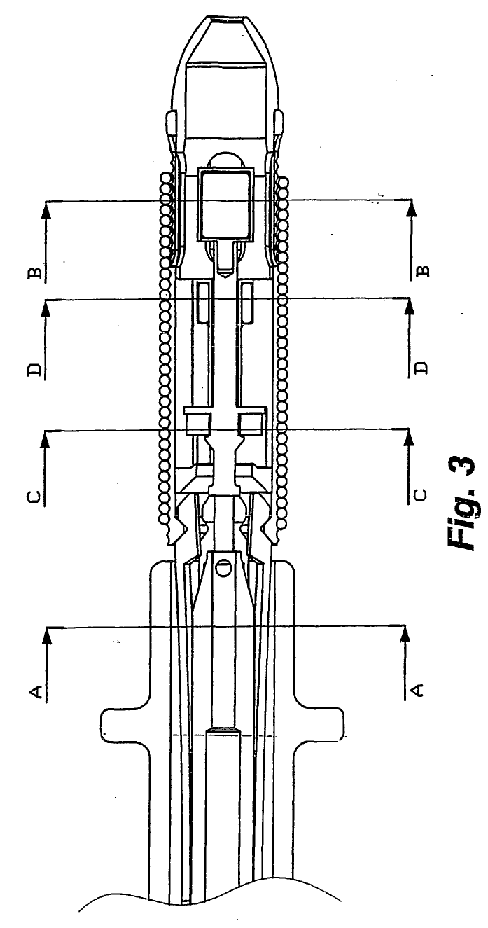

FIG. 4 radial sections A-D of the assembly of FIG. 3;

FIG. 5 a perspective view of the assembly of FIG. 3;

FIG. 6 the insertion device of FIG. 2, in a perspective view, in part enlarged;

FIG. 7 the sphincter of FIG. 1(C) and the frontal (proximal portion) of the insertion device of FIG. 2, aligned for coupling and in the same view;

FIG. 8 the combination of FIG. 7(C), coupled. And in the same view;

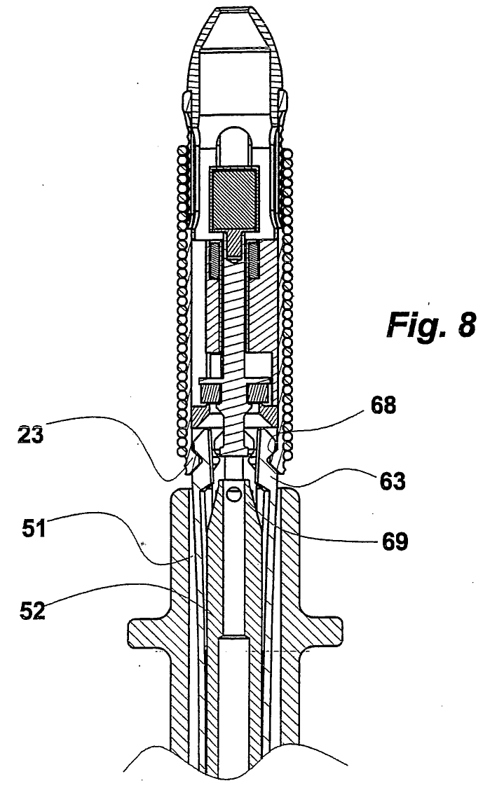

FIG. 9 the assembly of FIG. 8(C), inserted into an urethra of a female person and positioned for anchoring, in the same view;

FIG. 10 the assembly of FIGS. 8 and 9 (0), during injection of warm saline prior to expansion of the coil, in the same view;

FIG. 11 the assembly of FIGS. 8-10 (0), during injection of saline but after expansion of the shape memory metal coil, in the same view;

FIG. 12 the assembly of FIGS. 8-11 (C), after the end of injection of warm saline, in the same view;

FIG. 13 the combination of FIG. 12(C), after decoupling and separation of the sphincter and the insertion device, in the same view;

FIG. 14 the sphincter of FIG. 1(C), anchored in a female urethra, in the same view;

FIG. 15 a further embodiment of a sphincter valve assembly;

FIG. 16 is cross-sectional view of a valve assembly lifted by a catheter, open position;

FIG. 17 is cross-sectional view of the valve of FIG. 16 in closed position; and

FIG. 18 is a cross-sectional view with a catheter in position to flush a lower stent.

### DESCRIPTION OF PREFERRED EMBODIMENTS OF THE INVENTION

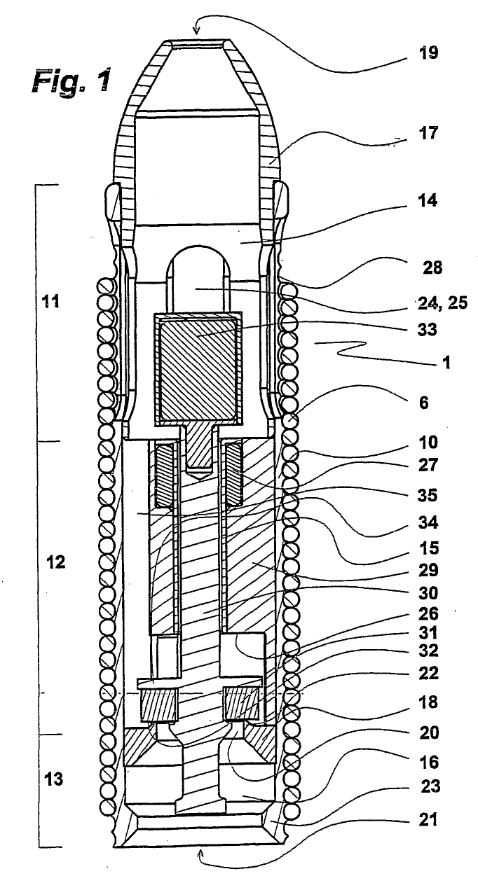

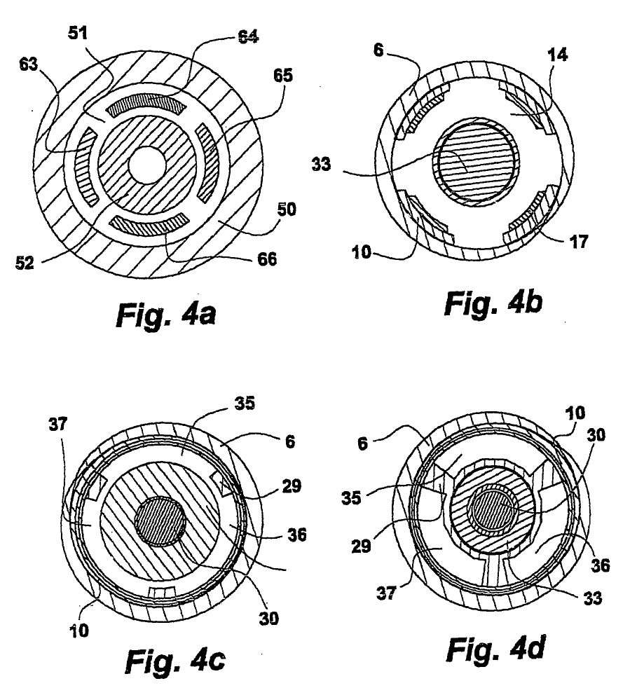

The artificial sphincter I of FIGS. 1 and 4b-4d is made of titanium or suitable polymer(s) or a combination thereof and comprises a valve body 10 of generally cylindrical shape provided with a central conduit extending from its distal end to its proximal end. In the following “axial” and “radial” should be understood as relating to the cylinder axis of the valve body 10; “proximal” and “distal” should be understood in relation to the disposition of the valve body 10 in a urethra 3. A portion of the outer mantle face of the valve body 10 is provided with shallow threads 28 of low pitch; the radial diameter of the valleys of the threaded portion is close to the radial distance between inner crests of a shape memory metal coil 6 mounted on the threads 28 of the valve body 10, so as to make the valve body 10 and the shape memory metal 6 coil abut each other. The pitch of the shallow threads 28 corresponds to the pitch of the shape memory metal coil 6. The valve body 10 comprises a proximal portion 11, a central portion 12, and a distal portion 13. The central conduit is comparatively wide in the terminal portions 11 and 13 so as to form proximal and distal chambers 14 and 16, respectively.

A roughly tubular proximal insert 17 is disposed in the proximal portion 11 of the valve body 10 from which it protrudes in a proximal direction so as to form the proximal end of the artificial sphincter 1. The inner wall of the proximal portion 11 of the valve body 10 and the distal portion of the tubular insert 17 are in abutment. The tubular insert 17 is fixed in the valve body 10 by a snap means not shown in the drawing. The proximal portion of 11 of the valve body 10 and the tubular insert 17 carry a number of superimposed draining slits 24 and 25, respectively, extending in an axial direction.

The distal chamber 16 comprises valve seat insert 18 dividing the chamber in a proximal portion and a distal portion. The valve seat insert 18 has a central bore 20 putting the aforementioned portions of the distal chamber in communication. On its proximal face the valve seat insert 18 carries a circular elevation 22 surrounding the bore 20. The radius of the elevation 22 is slightly larger than the -radius of the bore 20. Near the distal end of the body 10 the wall of the distal chamber 16 narrows to form a circular ridge 23.

In the central portion 12 of the valve body 10 is disposed a central insert 29 which, in combination with the inner wall of the central portion 12, defines three symmetrically disposed axially extending conduits 35, 36, 37 (FIG. 4d) set off radially from the cylinder axis by which chambers 14 and 16 are put in communication. A ring 27 of a ferromagnetic material is mounted around the proximal end portion of a central axial bore 15 provided in the central insert 29. An axially displaceable piston 30 is mounted in the central bore 15. Near its distal end the piston 30 carries a valve head ring 31 of a resilient material such as polyurethane, the distal face 32 of which capable of sealing against the valve seat elevation 22. The elevation 22 of the valve seat insert 18 and the distal face 32 of the valve head ring 31 constitute a valve means. The valve head ring 31 is supported by a radial flange 34 of the piston 30. A permanent magnet 33 is mounted at the proximal end of the piston 30.

In its extreme distal position of the piston 30 the magnet 33 is in Close proximity of the ferromagnetic ring 27, thereby keeping the piston 30 in this position and the distal face of the valve head ring 31 abuts the circular elevation 22. Thereby the artificial sphincter 1 is kept in a closed position (C). An extreme proximal position of the piston 30 is defined by the abutment of the proximal face of the flange 34 against a distal end face 26 of the central insert 29; the artificial sphincter 1 then is in an open position (O) allowing urine to be emptied from the bladder 4.

The shape memory metal coil of nickel-titanium alloy 6 comprises expandable 40, 41 (by martensite->austenite phase transformation) portions and a non-expandable 42 portion connecting the expandable portions 40, 41. The critical temperature of the expandable portions is 40-41° C. During expansion the expandable portion 40,41 are partially uncoiling whereby the length of the coil 6 is reduced while its diameter is increased in the expandable portions. When the shape memory metal coil 4 of the invention disposed in an urethra 3, is cooled to about 100 it becomes soft. A shape memory metal coil disposed in an urethra 3 can be withdrawn at that temperature from the urethra in form of a twisted string.

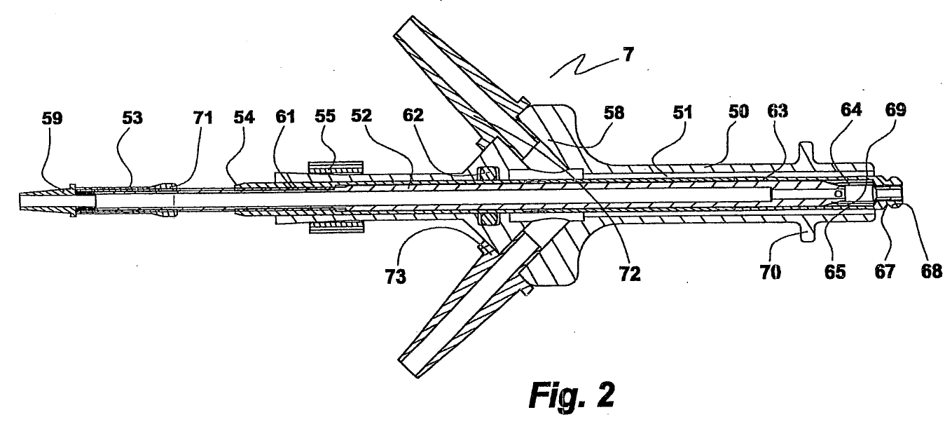

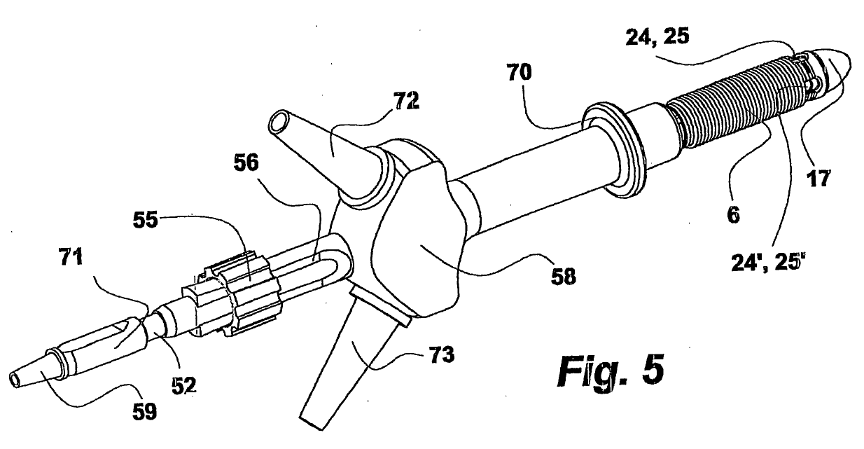

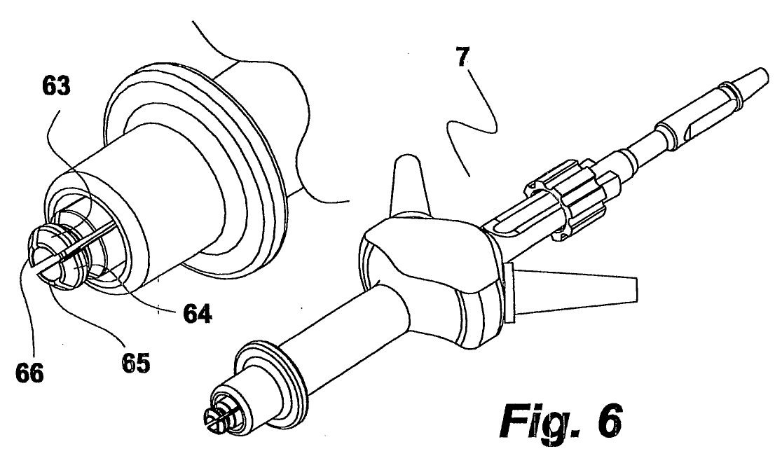

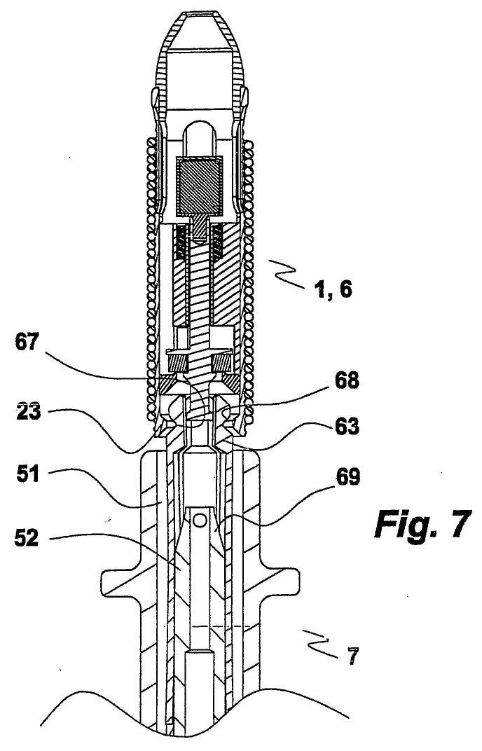

The insertion device 7 of the invention shown in FIGS. 2 and 4a, 5, 6 comprises an oblong body 50 with a through bore 51 in which a coupling assembly comprising four flexible stainless steel fingers 63, 64, 65, 66 disposed symmetrically in the through bore 51 between the walls thereof and a tubular coupling rod 52 by which the artificial sphincter I can be coupled with the insertion instrument 7 and released from it. Except for a central portion 58 the body 50 is essentially cylindrical. The coupling rod 52 has a truncated proximal end portion 69 (FIG. 7). The coupling rod 52 and the steel fingers 63, 64, 65, 66 are connected by cooperating threads 61 arranged at the outside of a distal portion 54 of the coupling rod 52 and at the inside of the fingers 63, 64, 65, 66 extending over a distance from their distal ends. Over a distal end portion 53 the coupling rod 52 has a sleeve grip 71 fixed to it.

The coupling assembly or parts thereof can be displaced in regard of the insertion instrument body 50 in two ways.

Firstly, the entire coupling assembly consisting of the coupling rod 52 and the fingers 63, 64, 65, 66 can be displaced by in a proximal direction (forwards) or a distal direction (backwards) in regard of the insertion device 7 by pushing or pulling, respectively, the grip 71. The fingers 63, 64, 65, 66 are resiliently flexible and have a shape that makes them fit into the through bore 51 without a need to compress them radially inwards. By the displacement of the entire coupling assembly their proximal ends can be moved from a position within the distal end portion of the insertion instrument body 50 to an outside position distally off said distal end portion.

Secondly, by turning the sleeve grip 71, the coupling rod 52 can be displaced over a short distance relative to the fingers 63, 64, 65, 66. By displacing the coupling rod 52 in this way in a distal direction its truncated cone portion 69 co-operates with inwardly narrowing portions of the steel fingers 63, 64, 65, 66 to displace their proximal ends slightly radially outwards. As will be explained below, this movement provides for coupling of the insertion instrument 7 with the artificial sphincter 1.

The coupling assembly 52, 63, 64, 65, 66 can be locked in any selected position in regard of the insertion instrument body 50 by a corrugated ring 55 threaded onto the distal end portion of the insertion instrument body 50 having walls which are slightly radially inwards displaceable by a pair of axially extending slits 56, 57 (only one shown) and which widen slightly radially towards their distal end. The distal end of the coupling rod 52 is provided with a terminal stud 59.

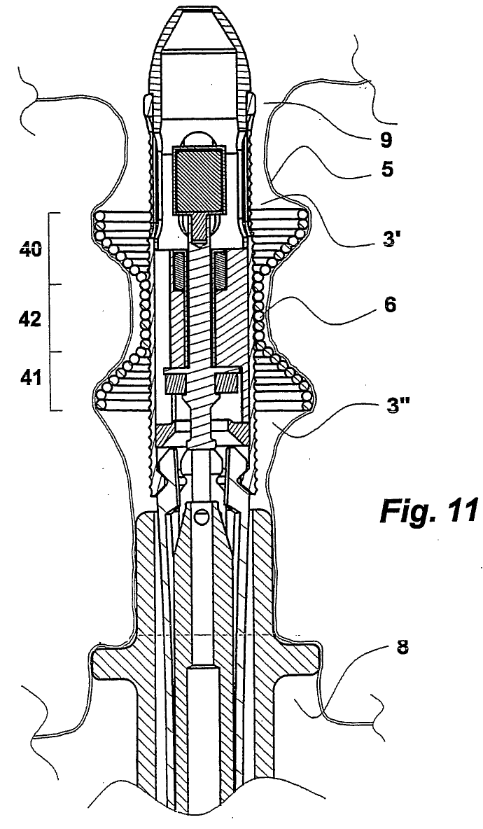

From the central portion 58 of the body 50 two studs 72, 73 extend obliquely in a distal direction. Flexible tubes for adducing or draining liquids can be coupled to the studs 59, 72, 73hich all are in communication with the through bore 51. A rubber ring 62 seals the distal portion of the through bore 51 from its the central and proximal portion which are intended to receive liquids. Near their proximal ends the fingers 63, 64, 65, 66 thicken whereby notches 67 are formed in their outer faces and shoulders 68 at their inner faces (FIG. 7). At a distance of a about 2 cm (device for female patients) or about 5 to 8 cm (device for male patients) from its proximal end the device body 50 is provided with an external circular flange 70 the purpose of which is to seal against the tissue surrounding the outer urethral opening 8 (FIG. 11).

The insertion of the artificial sphincter of FIGS. 1 and 4b-4d by means of the insertion device of FIGS. 2, 4a, 5, 6 will now be explained by reference to FIGS. 7 13.

FIG. 7 shows the sphincter 1 with the shape memory metal coil 6 mounted on it and the insertion device 7 disposed with their cylinder axes aligned prior to coupling. The front end portions of the fingers 63, 64, 65, 66 are inserted in the distal opening 21 of the artificial sphincter 1 to make their notches 67 face the circular ridge 23 of the valve body 10. The coupling rod 52 is in a withdrawn position to allow the tips of steel fingers 63, 64, 65, 66 assume their unloaded configuration.

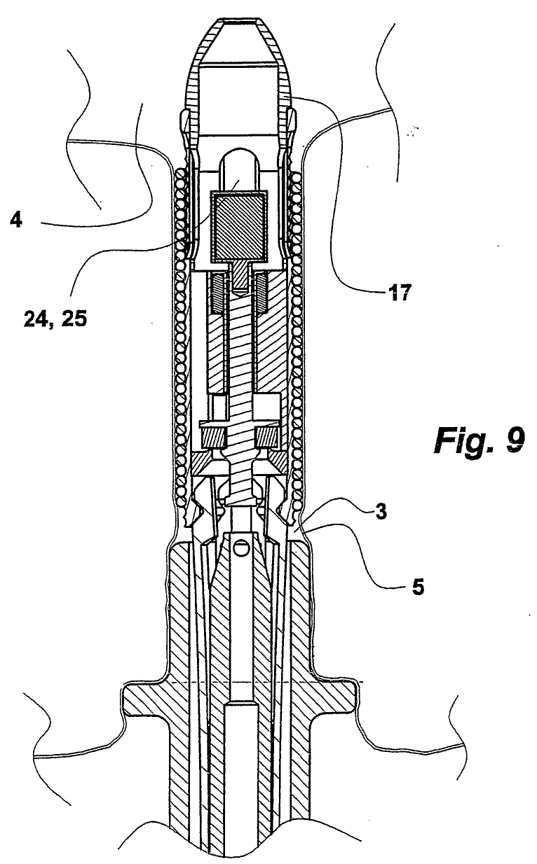

In the next step the sleeve 71 is rotated, whereby the coupling rod 52 is displaced towards the proximal end of the insertion instrument 7, whereby the slanting face of its truncated cone portion 69 abuts inwardly narrowing inner faces of the steel fingers 63, 64, 65, 66 near their distal ends and displaces them radially outwards to make them engage with the circular ridge 23. Thereby the insertion instrument 7 becomes firmly coupled to the artificial sphincter 1 (FIG. 9).

In the following step the artificial sphincter 1 is inserted with its proximal end first into a urethra 3 of a female person by means of the insertion device 7, a proximal portion of which is inserted to a depth to make the flange 70 seal against the tissue enclosing the outer urethral Opening 8. The insertion can be followed by medical imaging, such as, by X-ray or ultrasound imaging. Thereby the proper insertion depth can be controlled. Usually an insertion depth is selected in which the artificial sphincter 1 is disposed in the portion of the urethra 3 surrounded by the natural sphincter 2. Thereby its distal end portion up to including at least a proximal portion of the slits 24, 25 will extend into the bladder 4 so that free inflow of urine into the proximal chamber 14 and complete emptying of the bladder 4 is provided for (FIG. 9).

Insertion instruments of the invention of different design are used for male and female patients due to the difference in depth of insertion. In this respect the critical dimension is the distance from the distal end of the insertion instrument to the flange 34 which is used for sealing the outer urethral opening 8 during the following step.

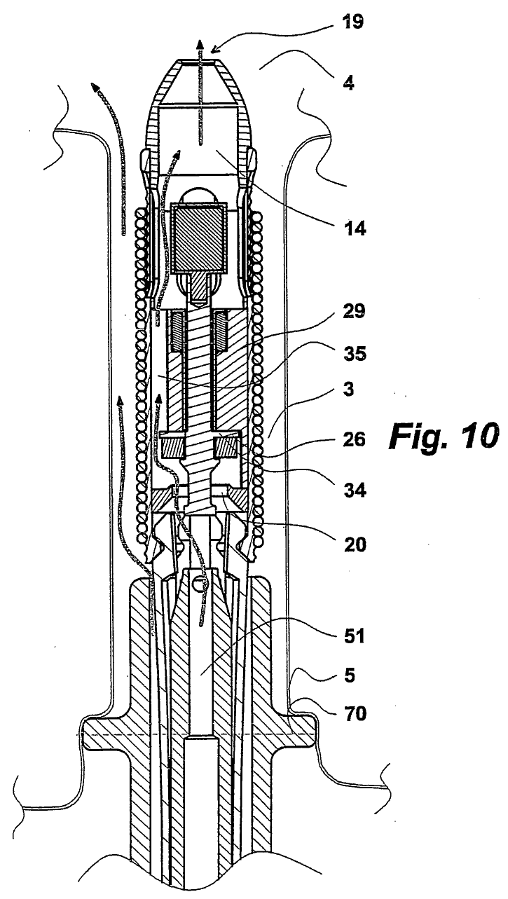

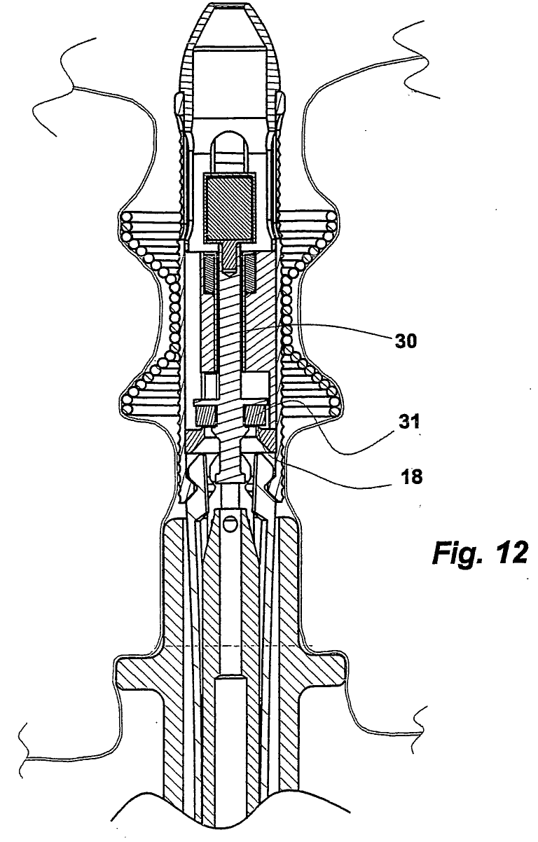

Next the shape memory metal coil 7 is made to partially expand by contacting it with a flow of saline of a temperature of about 45-48° C. (FIG. 10). The warm saline is forced into the insertion instrument 7 by the stud 72. From there it flows in a proximal direction in the space between the wall of the bore 51, the rod 62, and the fingers 63, 64, 65, 66. It then leaves the bore 61 at its distal end from where it flows inside and outside of the valve body 10 into the bladder 4. Due to the liquid pressure the valve 18, 22, 31, 32 opens and the piston 30 is displaced in a proximal direction. The flow of warm saline heats the shape memory metal coil 6 to a temperature above 410 C at which portions 40, 41 of the coil 6, which are capable of expansion, assume expanded conformations, whereas the central portion 42 connecting them, which is not capable of expansion, remains in its original conformation and keeps the coil 6 fastened to the valve body 10. By this partial expansion of the coil 6 which provides for intimate contact of the expanded sections with the urethral wall 5 the artificial sphincter I is sealingly anchored in the urethra 3 (FIG. 11). Thereby flow of liquid from the bladder 4 trough the inner urethral opening 9 via proximal 31 and distal 3″ portions of the urethra 3 is confined to flow through the sphincter 1 of the invention. At the end of the forced inflow of saline the valve 18, 32 closes (FIG. 12).

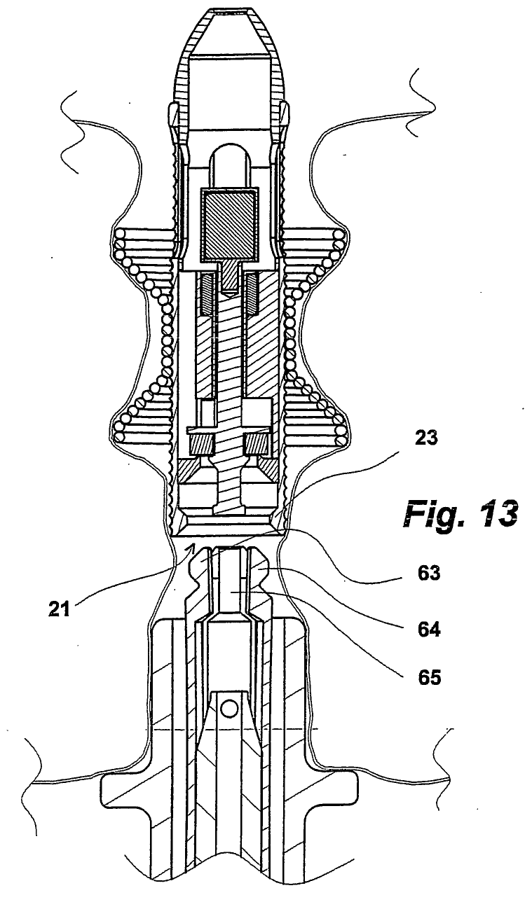

Finally the artificial sphincter 1 is released from the insertion device 7 by rotating the rod 52 in a direction opposite to the coupling direction (FIG. 13).

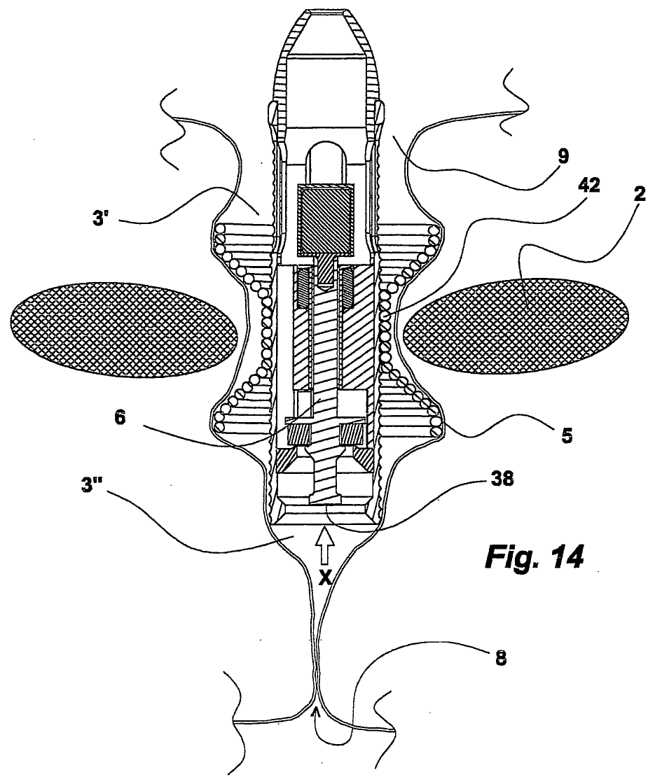

The insertion device 7 is withdrawn whereby the artificial sphincter 1 is left anchored in the urethra 3 ready for use (FIG. 14). The disposition of the sphincter in shown in FIG. 14 is a preferred one, the non-expandable portion 42 of the shape memory metal coil 6 being surrounded by natural sphincter 2, that is being situated in the plane defined by the natural sphincter 2. First of all, the bladder 4 will have to be emptied from the saline that accumulated during the anchoring step. This is achieved by inserting (direction indicated by arrow X in FIG. 14) a polished plastic rod with a rounded top (not shown) Into the urethra 3 from outside until it abuts the distal end face 38 of piston 30. By pushing the rod further in the same direction the piston 30 is displaced in a proximal direction whereby the valve 18, 32 is opened for outflow of the liquid in the bladder 4. Upon withdrawal of the rod the artificial sphincter I closes again and is kept in that position by the magnetic attraction of elements 27, 33. A person skilled in the art will easily understand that the present invention may also be applied to intra-urethral valves using different, for instance electromagnetic, valve mechanisms.

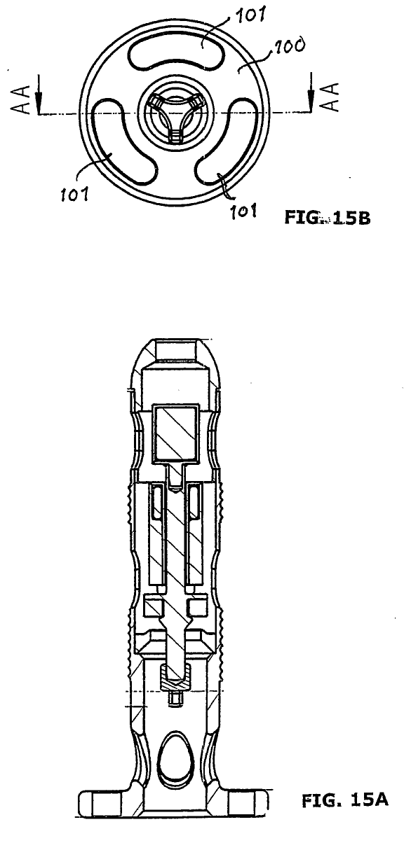

In a further embodiment shown in FIG. 15 the sphincter has a distal end provided with a flange 100. The flange is intended to be positioned outside the uretra opening preventing the device from being pushed into the bladder.

In this embodiment the distal half of the sphincter is equipped with one ore more holes 101 (FIG. 15B) positioned at least partly radially under the most distal shape memory coil of the sphincter, when this coil is not yet expanded. The holes connect the central canal of the sphincter with the outside of the sphincter and allow fluid to be flushed from the inside of the sphincter, through the holes and towards the surface of the coil, thus controlling the coil expansion of the distal coil.

In the method for Inserting an artificial sphincter of the invention encompasses flushing the shape memory coils of the sphincter in separate sequences.

In one sequence the most distal coil is flushed with temperated fluid. In this sequence the valve is closed preventing the fluid from going into the bladder, thus enabling a more efficient flushing of the distal coil as such.

In the other sequence the proximal and central coils are flushed with temperated fluid. In this sequence the valve is open allowing the fluid to reach these coils.

General Information of How to Handle the Sphincter at Insertion

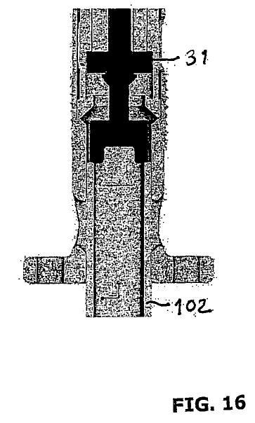

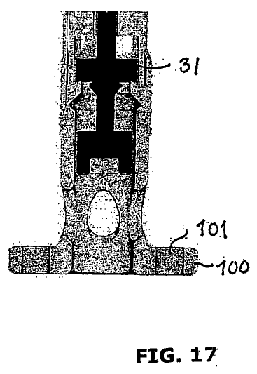

The valve is thus designed with a magnetic force to close; the valve closes with the urine flow (pressure). The valve is operated manually with an open-end catheter. To micturate, a catheter 102 is inserted in the valve and lifts the plunge to put the valve in an open position, see FIG. 16. The insertion length of the catheter is approximately 9 mm. This will lift the plunger 2 mm. Urine is drained through the catheter until the bladder is empty. When the catheter is removed the valve closes, see FIG. 17.

Insertion Procedure

Cleansing and Emptying the Patients Bladder

Prior to the insertion the patient's bladder should be flushed removing debris from the bladder floor. Flush with at least 200 ml ambient temperature saline solution. When emptying the bladder from debris, the patient should be placed in a vertical position before inserting the catheter in the urethra to the level of the bladder neck.

Pre Heating

The device is heated in a water bath that holds 35° C. This is to reduce the necessary increase of temperature to activate the stents when inserted in the patient. The stent material is specified to start phase transition at 38° C. so pre-heating to 35° C. ensures that the insertion procedure could be preformed without undesired activation of the stents.

Positioning

The device is inserted in the urethra with a firm grip on the outside guide. The device position is defined when the outside guide is in close contact with the urethral meatus.

Flushing Through the Valve

A gentle proximal pressure is applied to the guide while flushing the valve with 52° C. sterile water through an open-end catheter. When flushing through the valve it is made sure that the valve is in an open position. This is accomplished when the catheter is inserted in the valve approximately 9 mm. Provided that there is liquid in the bladder it will start to flow in the catheter when the valve is open. This flushing activates the upper stents.

Flushing the Outside of the Device

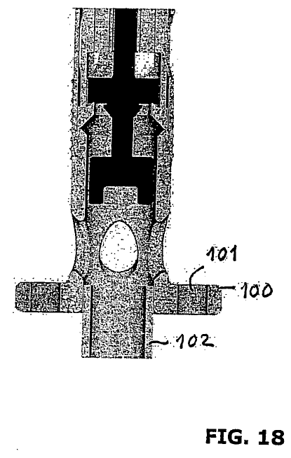

The outside of the device is flushed with 52° C. sterile water through an open-end catheter placed at the very entrance of the guide, see FIG. 18. When flushing the outside of the device it is made sure that the valve Is closed. Flushing the outside of the device is done to activate the lower stent and make sure that the upper stent is heated above the activation temperature. To confirm and document the insertion an X-ray should be performed.

If it is not possible to fully activate the stents the valve must be removed. If no bleeding occurs a new device can be inserted immediately thereafter.

Flushing

If leakage occurs because particles have got stuck in the device preventing closure of the valve, it may be possible that the next micturition will remove the particles. If not, it is necessary to flush the valve.

To flush:
An open-end catheter is inserted until the valve is fully open. The urine flow rate should increase when the valve is open.
The valve is flushed with 37° C. sterile water or saline solution.

The valve should always be flushed with 100 ml 37° C. sterile water or saline solution before considering removing the valve.

Removal of the Device

When a decision is made to remove the device prematurely from the patient the following procedure has to be carried out:
A. If leakage is the problem: locate the leakage, see below.
B. If possible, verify the position of the valve by ultra sound or x-ray. Insert an open-end catheter in the valve and open it. Flush ice-cold water through the catheter to cool the upper stents. Place the catheter in the lower position and flush with ice-cold water to cool the lower stent.

Use a forceps to grab the lower stent and pull out the device.

The stents needs to be soften prior to removal. If the stents are cooled below 8° C. the crystal structure transforms to a martensitic structure and softens. The wire feels like soldering wire in this condition. Please note that there is no shape transformation of the stent but it softens, so it is possible to remove the device from the patient without any trauma.

Localization of a Leakage

There is two possible flow channels for leakage of urine:
1. Leakage through the valve
2. Leakage between tissue and outside of the device.

To establish where the leakage occurs, the two flow channels need to be separated. This can be done by inserting an open-end catheter to a depth of 6,5 mm. This position requires accuracy when it comes to how far the catheter should be inserted. Mark the catheter at the distance 6,5 mm and insert it in the valve to the mark. If the catheter is inserted too far, an urine flow will be detected given that there is urine in the bladder. The catheter seals the lower flushing holes and leaves the valve in a closed position. There are now two separate flow cannels; one through the valve and catheter and a second between tissue and device outside the catheter.
1. The patient is asked to cough or laugh.
2. The leakage is observed to establish where it occurs.

### CONCLUSION

A. If the leakage only occurs through the catheter, leakage is most likely to occur through the valve.
B. If the leakage only occurs outside the catheter, the leakage is most likely between tissue and device.

1. An artificial sphincter comprising an anchoring element in form of a shape memory coil having a transition temperature, which is higher than the human body temperature of about 37° C., said artificial sphincter further comprising a valve body, which is at least partially enclosed by the shape memory metal coil and in a fixed relationship with it, and wherein the valve body is provided with a flange substantially perpendicular to its longitudinal axis, which flange is provided with at least one through-going hole.

2. The sphincter of claim 1, wherein the temperature is higher than 40° C.

3. The sphincter of claim 1, wherein the temperature is from 41° to 45° C.

4. The sphincter if claim 1, wherein the coil of shape memory metal comprises one or several sections which are capable of expanding at the transition temperature and one or several sections which are not capable of expanding at that temperature.

5. The sphincter of claim 4, wherein a coil section capable of expansion is of a nickel-titanium alloy.

6. The sphincter of claim 4, wherein the coil comprises two expandable sections separated by a non-expandable section.

7. The sphincter of claim 1, wherein the valve body is of a generally cylindrical shape and of a diameter which equals about the inner diameter of at least a section of the shape memory metal coil.

8. The sphincter of claim 7, wherein the exterior mantle face of the valve body comprises threaded helical groves having a pitch, which corresponds to the pitch of the shape memory metal coil.

9. The sphincter according to claim 1, wherein the plunge is arranged to being lifted by a catheter introduced into the valve body.

10. A method for inserting an artificial sphincter comprising a coil of shape memory metal into an urethra and anchoring it there, comprising coupling the sphincter including a shape memory metal coil comprising a coil section having a transition temperature above the normal human body temperature of 37° C. to an insertion device, inserting the sphincter by means the insertion device into a desired position in the urethra, adducing an aqueous fluid of a temperature higher than the transition temperature of said coil section through said insertion device to the coil to make it expand, thereby anchoring the coil with the sphincter in the urethra; releasing the sphincter from the insertion device; withdrawing the insertion device from the urethra.

11. A method for inserting an artificial sphincter comprising a coil of shape memory metal into an urethra and anchoring it there, comprising the following steps: providing an artificial sphincter of about cylindrical form comprising a coil of shape memory metal having a transition temperature above the normal human body temperature of 37° C. and being adapted at least partially surround and hold the sphincter;
coupling a distal end portion of the sphincter ot a coupling means disposed at the proximal end of an oblong insertion means having a proximal end portion of about cylindrical form which comprises an axial channel for transport of fluid, the coupling means thereby releasably holding the sphincter in a disposition in which the axes of the sphincter and the valve body coincide; inserting the sphincter and the proximal portion of the insertion means into the urethra, thereby disposing the sphincter in a desired position in the urethra; adducing an aqueous fluid into the urethra by said channel so as to make the fluid flow along the urethral wall and past the sphincter into the bladder, the fluid having a temperature above the critical point of the shape memory metal coil, thereby making a portion or portions of the coil expand in a radial direction; releasing the insertion means from the sphincter; and withdrawing the insertion means.

12. A device for insertion and anchoring of an artificial sphincter in an urethra, comprising a proximal end portion of about cylindrical shape, means for coupling with the sphincter of claim 1 disposed at the proximal end of said proximal end portion and being operable from a distal end portion thereof, a channel disposed within said proximal end portion and having an opening at the proximal end thereof for adducing and aqueous fluid of a temperature above the normal human body temperature of about 37° C. to the shape memory coil comprised by the artificial sphincter.

13. The device of claim 12, wherein the coupling means comprises two or more flexible fingers disposed in said channel.

14. The device of claim 13, wherein proximal portions of said fingers are independently displaceable in an axial direction and in a radial direction.

15. The device of claim 14, wherein said proximal finger portions can be displaced in an axial direction from a position within the channel to a position proximally outside of the channel.

16. The device of claim 14, wherein the radial displacement of said proximal finger portions is arranged for in their position proximally outside of the channel.

17. The device of claim 12, wherein the means for operation of the coupling means comprises a rod disposed in the channel.

18. The device of claim 13, wherein the means for operation of the coupling means comprises a rod disposed in the channel radially inwardly of said flexible fingers.

19. The device of claim 17, wherein the rod comprises a slanting face disposed at its proximal end.
